(19) United States
(12) Patent Application Publication
(10) Pub. No.: US 2026/0234029 A1
(43) Pub. Date: Aug. 13, 2026

(54) ULTRAPURE WATER PRODUCTION APPARATUS, METHOD OF PRODUCING ULTRAPURE WATER, METHOD OF MANAGING OPERATION OF THE ULTRAPURE WATER PRODUCTION APPARATUS, AND METHOD OF MEASURING COLLOIDAL PARTICLES

(71) Applicant: Nomura Micro Science Co., Ltd., Atsugi-shi (JP)

(72) Inventors: Masamitsu IIYAMA, Atsugi-shi (JP); Hiroki MIYAZAWA, Atsugi-shi (JP); Yuya HIRAHARA, Atsugi-shi (JP); Yukio NOGUCHI, Atsugi-shi (JP)

(73) Assignee: Nomura Micro Science Co., Ltd., Atsugi-shi (JP)

(21) Appl. No.: 19/530,445

(22) Filed: Feb. 5, 2026

(30) Foreign Application Priority Data
Feb. 12, 2025 (JP) 2025-020996

(51) Int. Cl. C02F 1/00; B01D 19/00; B01D 61/14; C02F 1/20; C02F 1/32; C02F 1/42; C02F 1/44; C02F 9/00; C02F 101/30; C02F 103/04

(52) U.S. Cl. CPC C02F 1/008; B01D 19/0031; B01D 61/145; C02F 9/00; C02F 1/20; C02F 1/32; C02F 1/42; C02F 2001/427; C02F 1/444; C02F 2101/30; C02F 2103/04; C02F 2209/20; C02F 2303/04

(57) ABSTRACT

The ultrapure water production apparatus includes a secondary pure water device including: a water supply pump, an ion exchanger, and an ultrafiltration membrane device in order of the water supply pump, the ion exchanger, and the ultrafiltration membrane device, the secondary pure water device being configured to remove an impurity from primary pure water, in which at least one of a first combination of an acoustic particle counter and an optical particle counter or a second combination of the acoustic particle counter and a total organic carbon (TOC) meter is disposed at least one of an upstream side or a downstream side of the ultrafiltration membrane device.

### RELATED APPLICATION(S)

This application claims the benefit of priority of Japanese Patent Application No. 2025-020996 filed on Feb. 12, 2025, the contents of which are incorporated by reference as if fully set forth herein in their entirety.

### FIELD AND BACKGROUND OF THE INVENTION

The technology of the present disclosure relates to an ultrapure water production apparatus, a method of producing ultrapure water, a method of managing operation of the ultrapure water production apparatus and a method of measuring colloidal particles.

### RELATED ART

Ultrapure water to be used in semiconductor production processes is produced with an ultrapure water production apparatus including a primary pure water device and a secondary pure water device in this order. In the ultrapure water production apparatus, the primary pure water device removes total organic carbon (TOC) components and ionic components in raw water or pretreatment water using a reverse osmosis membrane device or an ion exchanger to produce primary pure water. The secondary pure water device removes an extremely small amount of impurities in the primary pure water to produce ultrapure water. Further, it is typical that an ultrafiltration membrane (UF) device is installed at an end of the secondary pure water device to remove nanometer-sized particles.

In such an ultrapure water production apparatus, a particle counter for measuring the number of particles in water is disposed at the downstream side of the ultrafiltration membrane device, and the water quality is managed based on the measurement value of the particle counter (see, for example, International Publication Pamphlet No. 2015/064628). International Publication Pamphlet No. 2015/064628 describes a configuration in which a plurality of optical particle counters is disposed for improvement in the accuracy of measuring particles. Japanese Patent Publication (JP-B) No. 7563959 describes an ultrapure water production apparatus that controls the flow rate of treatment-target water circulating in the ion exchanger for efficient removal of the particles.
Patent Document 1: International Publication Pamphlet No. 2015/064628
Patent Document 2: Japanese Patent Publication (JP-B) No. 7563959

### SUMMARY OF THE INVENTION

In semiconductor production processes, ultrapure water is used for cleaning a semiconductor wafer. An adherence of impurities contained in the ultrapure water to the semiconductor wafer may adversely affect the quality of semiconductor. In recent years, with the remarkable progress of miniaturization and high integration of semiconductor circuits, the required water quality for the ultrapure water used in the semiconductor production processes has become increasingly severe.

The impurities adhering to the semiconductor wafer contain colloidal particles in addition to particles. The colloid refers to a state in which particles much smaller than particles are suspended like a cloud while being dispersed in liquid and having a certain degree of aggregation. Here, the aggregation of particles in such a state is referred to as colloidal particles.

In an ultrapure water production apparatus, various sources of colloidal particles are conceivable, but one of the sources is considered as an ion exchanger. For example, the bonds of unstable functional groups are broken from the surface of the ion exchange resin. The broken functional groups are desorbed from the ion exchange resin and aggregated, or aggregated on the surface of the ion exchange resin, thereby forming colloidal particles, which are suspended in water.

Unlike ions, the colloidal particles do not completely dissolve into liquid, and have both properties of non-particulate properties such as ions and particulate properties such as particles. The colloidal particles may be considered to be smaller in particle size than the particles. Because the colloidal particles are not perfect particles each having a clear outer edge, and each have a very low optical refractive index as compared with the particles. Thus, it has been difficult to measure the optical refractive index with a known optical particle counter.

However, it is known that the presence of colloidal particles adhering to a semiconductor wafer can be confirmed with a scanning electron microscope (SEM), and that the colloidal particles may adversely affect the quality of semiconductor as described above. Therefore, in the processes of producing ultrapure water, it has been desired to measure colloidal particles such that the colloidal particles in the treatment-target water are quantitatively grasped or at least the tendency of increase or decrease is grasped.

The technology of the present disclosure provides an ultrapure water production apparatus that can be used for measuring colloidal particles, a method of producing ultrapure water, a method of managing operation of the ultrapure water production apparatus, and a method of measuring the colloidal particles.

An ultrapure water production apparatus according to the technology of the present disclosure is an ultrapure water production apparatus including a secondary pure water device including: a water supply pump, an ion exchanger, and an ultrafiltration membrane device in this order, the secondary pure water device being configured to remove an impurity from primary pure water, in which at least one of a first combination of an acoustic particle counter and an optical particle counter or a second combination of the acoustic particle counter and a total organic carbon (TOC) meter is disposed at at least one of an upstream side or a downstream side of the ultrafiltration membrane device.

The ultrapure water production apparatus according to the technology of the present disclosure may include a processor configured to control a water recovery rate of the ultrafiltration membrane device.

A method of producing ultrapure water according to the technology of the present disclosure is a method of producing ultrapure water using the ultrapure water production apparatus.

A method of managing operation of an ultrapure water production apparatus according to the technology of the present disclosure is a method of managing an operation of an ultrapure water production apparatus including a secondary pure water device including: a water supply pump, an ion exchanger, and an ultrafiltration membrane device in this order, the secondary pure water device being configured to remove an impurity from primary pure water, the method comprising: using at least one of a first combination of an acoustic particle counter and an optical particle counter or a second combination of the acoustic particle counter and a total organic carbon (TOC) meter, the at least one of the first or second combination being disposed at a downstream side of the ultrafiltration membrane device; and managing operation of the ultrapure water production apparatus, based on measurement values acquired from the at least one of the first or second combination.

A method of measuring colloidal particles according to the technology of the present disclosure is a method of measuring colloidal particles in a secondary pure water device including: a water supply pump, an ion exchanger, and an ultrafiltration membrane device in this order, the secondary pure water device being configured to remove an impurity from primary pure water, the method including: using at least one of a first combination of an acoustic particle counter and an optical particle counter or a second combination of the acoustic particle counter and a total organic carbon (TOC) meter, the at least one of the first or second combination being disposed at a downstream side of the ultrafiltration membrane device; and measuring colloidal particles, based on measurement values acquired from the at least one of the first or second combination.

According to the technology of the present disclose, provided can be an ultrapure water production apparatus that can be used for measuring colloidal particles, a method of producing ultrapure water, a method of managing operation of the ultrapure water production apparatus, and a method of measuring the colloidal particles.

### BRIEF DESCRIPTION OF THE SEVERAL VIEWS OF THE DRAWING(S)

FIG. 1 illustrates the schematic configuration of an ultrapure water production apparatus;

FIG. 2 illustrates the schematic configuration of a secondary pure water device;

FIG. 3 is a schematic view conceptually illustrating colloidal particles;

FIG. 4A is a schematic view conceptually illustrating the principle of an optical particle counter;

FIG. 4B is a schematic view conceptually illustrating the principle of the optical particle counter;

FIG. 5A is a schematic view conceptually illustrating the principle of an acoustic particle counter;

FIG. 5B is a schematic view conceptually illustrating the principle of the acoustic particle counter;

FIG. 6 is a graph indicating the experimental result of the optical particle counter;

FIG. 7 is a graph indicating the experimental result of the acoustic particle counter;

FIG. 8A illustrates the comparison of characteristics between the acoustic particle counter and the optical particle counter;

FIG. 8B illustrates the comparison of characteristics between the acoustic particle counter and the optical particle counter;

FIG. 9 illustrates the combination of the acoustic particle counter and the optical particle counter;

FIG. 10A schematically illustrate the states in which particles and colloidal particles are captured on a filtration membrane;

FIG. 10B schematically illustrate the states in which particles and colloidal particles are captured on the filtration membrane;

FIG. 10C schematically illustrate the states in which particles and colloidal particles are captured on the filtration membrane;

FIG. 11 is a graph indicating examples in which colloidal particles are removed by changing a water recovery rate;

FIG. 12 illustrates the combination of the acoustic particle counter and a total organic carbon (TOC) meter;

FIG. 13A illustrates the comparison of characteristics between the acoustic particle counter and the TOC meter; and

FIG. 13B illustrates the comparison of characteristics between the acoustic particle counter and the TOC meter.

### DESCRIPTION OF SPECIFIC EMBODIMENTS OF THE INVENTION

An exemplary embodiment of the present disclosure will be explained below. However, the present disclosure is not limited to the following exemplary embodiment. When the exemplary embodiment is explained in the present disclosure with reference to the drawings, the configuration of the exemplary embodiment is not limited to the configuration shown in the drawings. Furthermore, sizes of members in the drawings are conceptual, and relative relationships between the sizes of the members are not limited thereto.

In the following description of the drawings, the same or similar parts are denoted by the same or similar reference numerals. However, the drawings are schematic, and relationships between thicknesses and planar dimensions, ratios between thicknesses of respective apparatuses or respective members, and the like are different from those in actuality. Therefore, the specific thicknesses and planar dimensions should be determined in consideration of the following explanation. Further, the drawings include portions for which the dimensional relationships or ratios are different from each other between drawings. Furthermore, unless otherwise specified in the specification, the numbers of respective constituent elements of the present disclosure are not limited to one, and a plurality of each of the constituent elements may be present.

In the following exemplary embodiment, the constituent elements thereof (including element steps and the like) are not essential unless otherwise specified. The same also applies to numerical values and ranges thereof, and the present disclosure is not limited thereto. In numerical ranges indicated using “to” in the present disclosure, numerical values described before and after “to” are respectively included as a minimum value and a maximum value.

In numerical ranges described in a stepwise manner in the present disclosure, an upper limit value or a lower limit value described in one numerical range may be replaced with an upper limit value or a lower limit value of another numerical range described in a stepwise manner. Furthermore, in numerical ranges described in the present disclosure, an upper limit value or a lower limit value of the numerical range may be replaced with a value indicated in the examples.

In the present disclosure, when components are contained, each component may include plural kinds of substances corresponding thereto. When plural kinds of substances corresponding to a respective component are present in a composition, a content ratio or content of each component means, unless otherwise specified, a total content ratio or content of the plural kinds of substances present in the composition.

In the present disclosure, particles corresponding to respective components may include plural kinds of particles. When plural kinds of particles corresponding to a respective component are present in a composition, a particle size of each component means, unless otherwise specified, a value for a mixture of the plural kinds of particles present in the composition.

In the present disclosure, the term “layer” or “membrane” includes not only cases in which, when a region in which the layer or membrane is present is observed, the layer or membrane is formed in an entirety of the region, but also cases in which the layer or membrane is formed only in a portion of the region.

### [Ultrapure Water Production Apparatus]

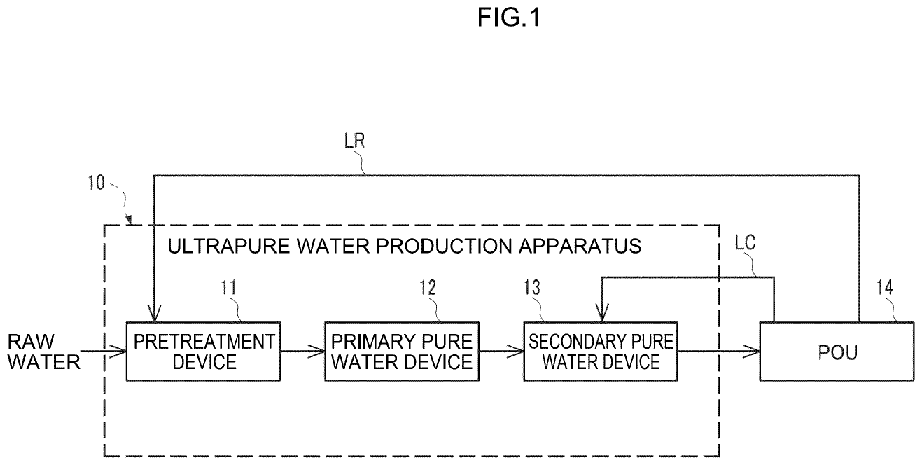

Hereinafter, an ultrapure water production apparatus according to the technology of the present disclosure will be described. As illustrated in FIG. 1, an ultrapure water production apparatus 10 according to the present embodiment includes a pretreatment device 11, a primary pure water device 12, and a secondary pure water device 13. The ultrapure water produced by the ultrapure water production apparatus 10 is sent to a point of use (POU) 14 and used at the POU 14. The POU 14 is, for example, a semiconductor production apparatus and the ultrapure water is used for cleaning a semiconductor wafer in the semiconductor production processes.

Among the ultrapure water sent to the POU 14, unused ultrapure water is returned to the secondary pure water device 13 through a circulation line LC. Although the single POU 14 is illustrated in FIG. 1, the ultrapure water production apparatus 10 may supply the ultrapure water to two or more pieces of POU 14. The used ultrapure water having been used at the POU 14 is returned to the pretreatment device 11 through a recovery line LR. The circulation line LC and the recovery line LR are pipelines through which liquid flows, and are configured by piping.

### (Pretreatment Device)

Raw water is supplied to the pretreatment device 11. The pretreatment device 11 includes a flocculation and sedimentation device, a sand filtration device, and a membrane filtration device. The flocculation and sedimentation device, the sand filtration device, and the membrane filtration device each perform clarification to the raw water, whereby pretreatment water from which some of suspended substances and organic substances have been removed is produced. As the raw water, industrial water, tap water, groundwater, or river water can be used. If a urea removing device including a combination of a high pressure reverse osmosis membrane device, addition of hypobromous acid, and an ultraviolet irradiator, or a high pressure reverse osmosis membrane device and a boron removing device such as a boron-selective ion exchanger are additionally provided to the pretreatment device 11, brackish water or sewage can also be used as the raw water. As the raw water, one of these may be used, or two or more thereof may be used in combination.

### (Primary Pure Water Device)

The primary pure water device 12 performs a primary pure water treatment for converting the raw water into primary pure water. The primary pure water device 12 performs a cleaning treatment to the raw water, removes impurities from the raw water, and produces primary pure water. Specifically, the primary pure water device 12 includes various devices such as a desalinator for removing impurity ions, a reverse osmosis membrane device for removing inorganic ions, organic substances, and particles, a vacuum degassing device or a membrane degassing device for removing dissolved gas such as dissolved oxygen, a regenerative mixed bed desalinator for removing remaining ions, and an electric deioninator. The pretreatment water may be subjected to the primary pure water treatment, instead of the raw water.

### (Secondary Pure Water Device)

The secondary pure water device 13 is a device that performs a secondary pure water treatment of removing an extremely small amount of impurities in the primary pure water to produce ultrapure water.

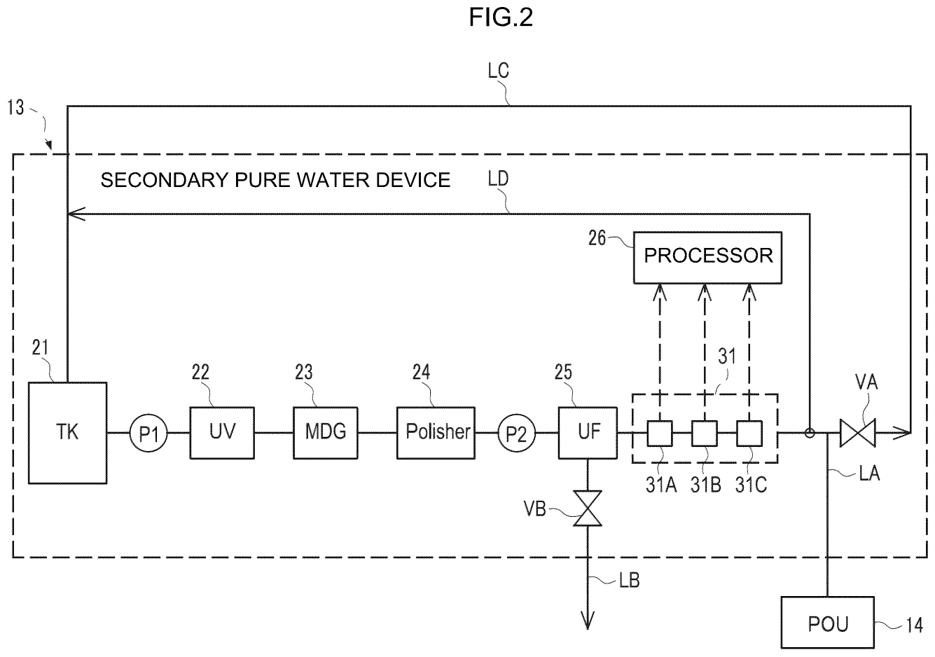

As illustrated in FIG. 2, the secondary pure water device 13 includes a pure water tank (TK) 21, a water supply pump P1 provided at the following stage of the pure water tank 21, an ultraviolet irradiator (UV) 22, a membrane degassing device (MDG) 23, a non-regenerative mixed bed ion exchange resin device (Polisher) 24, a booster pump P2, and an ultrafiltration membrane device (UF) 25 in this order. In the secondary pure water device 13, the primary pure water stored in the pure water tank 21 is sequentially supplied, by the water supply pump P1 and the booster pump P2, to each of the devices from the ultraviolet irradiator 22 to the ultrafiltration membrane device 25 that are provided at the following stage of the pure water tank 21. The supplied primary pure water is subjected to treatment in each of the devices, whereby ultrapure water is produced. The booster pump P2 may be provided at the previous stage of the non-regenerative mixed bed ion exchange resin device 24.

The material and shape of the pure water tank 21 are not particularly limited as long as there is no generation of rust, there is almost no elution of components from the container, and the primary pure water can be stably stored. The pure water tank 21 is preferably made of fiber reinforced plastics (FRP), polyethylene, and SUS304 or a material obtained by lining with, for example, a fluororesin such as TEFLON (trademark).

### (Ultraviolet Irradiator)

The ultraviolet irradiator 22 irradiates the primary pure water with ultraviolet light to decompose an organic substance and kill (that is to say, sterilize) live bacteria in the primary pure water. The ultraviolet irradiator 22 includes, for example, an ultraviolet lamp capable of emitting ultraviolet light having a wavelength of approximately 185 nm or a wavelength of approximately 254 nm, and can reliably decompose the organic substance and sterilize the live bacteria in the primary pure water. The ultraviolet lamp of the ultraviolet irradiator 22 is not particularly limited, but a low-pressure mercury lamp is preferable in terms of ease of handling. Examples of the ultraviolet irradiator 22 include a circulation type in which an ultraviolet lamp is disposed along a flow path of treatment-target water inside the housing and an immersion type in which an ultraviolet lamp is immersed in a tank that stores treatment-target water, but the circulation type is preferable from the viewpoint of treatment efficiency.

### (Membrane Degassing Device)

The membrane degassing device 23 removes a gas, particularly dissolved oxygen, in the primary pure water using a gas separation membrane that does not permeate moisture but permeates gas.

### (Non-Regenerative Mixed Bed Ion Exchange Resin Device)

The primary pure water from which the dissolved oxygen concentration has been removed by the membrane degassing device 23 is fed to the non-regenerative mixed bed ion exchange resin device 24. The non-regenerative mixed bed ion exchange resin device 24 adsorbs and removes an organic acid generated by decomposition of the organic substance by the ultraviolet irradiator 22 and impurity ions such as metal ions remaining in the water. The non-regenerative mixed bed ion exchange resin device 24 has, for example, a structure in which a cylindrical sealed container is filled with a mixed bed ion exchange resin. The mixed bed ion exchange resin is usually a mixture of a cation exchange resin and an anion exchange resin. The non-regenerative mixed bed ion exchange resin device 24 is an example of an “ion exchanger” of the present disclosure.

### (Booster Pump)

The booster pump P2 is a pump that supplementarily applies pressure to increase the water pressure. The water pressure on the outlet side of the ultrafiltration membrane device 25 is secured by the booster pump P2, whereby the water pressure required at the POU 14 can be appropriately maintained.

### (Ultrafiltration Membrane Device)

The ultrafiltration membrane device 25 includes one ultrafiltration membrane module or a plurality of ultrafiltration membrane modules or includes one ultrafiltration membrane cartridge or a plurality of ultrafiltration membrane cartridges. The ultrafiltration membrane modules each include a housing having a water inlet for supply, an outlet for permeated water, and an outlet for concentrated water, and an ultrafiltration membrane is provided inside the housing. The respective openings of the modules are connected to a pipe, whereby the treatment-target water passes through the ultrafiltration membrane. The ultrafiltration membrane modules are advantageous in that when the ultrafiltration membranes are clogged and the filtration performance is deteriorated, the modules are replaced and the deteriorated ultrafiltration membranes can be cleaned for each module. The ultrafiltration membrane installed in each of the ultrafiltration membrane modules may also be referred to as an ultrafiltration membrane cartridge. For the ultrafiltration membrane cartridge, only the cartridge in the ultrafiltration membrane module can be replaced. The ultrafiltration membrane includes, for example, a hollow fiber membrane.

The ultrafiltration membrane device 25 removes particles in water, for example, particles each having a particle size of 50 nm or more, preferably 10 nm or more.

The ultrafiltration membrane device 25 includes an ultrafiltration membrane module or a plurality of ultrafiltration membrane modules. The type of the ultrafiltration membrane included in the ultrafiltration membrane module is not particularly limited, and the ultrafiltration membrane is usually a hollow fiber membrane, and may be a spiral membrane, a tubular membrane, or a flat membrane. The material of the ultrafiltration membrane is polysulfone, polyvinylidene fluoride, polyethylene, or polypropylene, and the nominal molecular weight cutoff is preferably 3000 to 8000 and more preferably 4000 to 6000. As the ultrafiltration membrane module, either an internal pressure type or an external pressure type may be used. The ultrafiltration membrane device 25 may be of a cross-flow type or a total filtration type. The ultrafiltration membrane device 25 may have a plurality of stages in series.

In the ultrafiltration membrane device 25, a water inlet is connected to a water passage line on the booster pump P2 side, a permeation-side outlet is connected to the circulation line LC, and a concentration-side outlet is connected to a water drain line LB. A valve VA is provided in the path of the circulation line LC. The circulation line LC branches on the upstream side of the valve VA, and a branched water supply line LA is connected to the POU 14. The ultrapure water not used at the POU 14 is returned to the pure water tank 21.

A measurement device 31 for measuring impurities in the treatment-target water is disposed in the pipeline on the downstream side of the ultrafiltration membrane device 25. As an example, the measurement device 31 includes an acoustic particle counter 31A, an optical particle counter 31B, and a TOC meter 31C. A measurement value of the measurement device 31 is output to a processor 26. The processor 26 manages the water quality based on the measurement value.

The treatment-target water is subjected to impurity removal in the ultrafiltration membrane device 25, but the impurities cannot be completely removed even by the ultrafiltration membrane device 25. Based on the measurement value acquired from the measurement device 31, the processor 26 monitors whether or not the amount of impurities contained in the treatment-target water is within a preset allowable range. Then, the processor 26 repeats the removal of impurities by the ultrafiltration membrane device 25 and the others until the measurement value falls within the allowable range, and sends the produced ultrapure water to the POU 14 in a case where the measurement value falls within the allowable range. That is, a bypass line LD returning to the pure water tank 21 is connected to the downstream side of the measurement device 31 before branching to the water supply line LA. A valve (not illustrated) can switch the destination of the treatment-target water having passed through the ultrafiltration membrane device 25 between the bypass line LD and the water supply line LA. In a case where the water quality is out of the allowable range, the treatment-target water in the ultrafiltration membrane device 25 is directly returned to the pure water tank 21 through the bypass line LD returning to the pure water tank 21, and when the measurement value is within the allowable range, the valve switches the destination of the treatment-target water to the water supply line LA for supply to the POU 14. The impurities contain, in addition to particles, dissolved organic carbon (DOC) dissolved in the treatment-target water, colloidal particles, metals such as iron, copper, and calcium, and boron.

The acoustic particle counter 31A sends a sound wave having energy unique to the detection-target particles into the treatment-target water, and detects, with a piezoelectric element, a pressure of cavitation caused by the particles received the sound wave. Then, in a case where a pressure equal to or higher than a certain pressure is detected, the particles are counted, and the counted number of particles is output to the processor 26 as a measurement value. As the acoustic particle counter 31A, for example, “NanoPULS” (trademark) manufactured by OVIVO Inc. is used.

The optical particle counter 31B emits laser light into the treatment-target water, collects the scattered light obtained by scattering the laser light emitted to the particles by a condensing optical system, and detects the collected light by a photoelectric conversion element. Then, the optical particle counter 31B counts the number of particles based on the amount of received light having been detected, and outputs the counted number of particles to the processor 26 as a measurement value. As the optical particle counter 31B, for example, “Ultra DI (registered trademark) 20 Plus” manufactured by Particle Measurement Systems is used.

The TOC meter 31C is a measurement device that measures the total amount of carbon (total organic carbon: TOC) present in organic substances.

The total amount of carbon includes, in addition to the amount of dissolved organic carbon (DOC), the amount of particulate or colloidal organic carbon (that is to say, the amount of organic carbon that is not dissolved in the treatment-target water) although the proportion is small. As is well known, the TOC meter 31C decomposes an organic substance in the treatment-target water by ultraviolet irradiation or using an oxidizing agent, and measures the amount of generated carbon dioxide (CO₂) by an infrared absorption method or a conductivity method. Because the amount of generated carbon dioxide is proportional to the total amount of carbon contained in the organic substances, the total amount of carbon contained in the treatment-target water can be quantified by measuring the amount of generated carbon dioxide. The TOC meter 31C outputs the total amount of measured carbon as a measurement value. As the TOC meter 31C used here, any TOC meter for ultrapure water can be used without particular limitation, such as ANATEL A1000 XP manufactured by Beckman Coulter, Inc., ACCURA-SX II manufactured by T&C Technical, Sievers M500e, Sievers 500RL, and Sievers 900 manufactured by SUEZ, and 6000TOC manufactured by METTLER TOLEDO.

The processor 26 can measure colloidal particles, which are impurities in the treatment-target water, based on the measurement value of the acoustic particle counter 31A and the measurement value of the optical particle counter 31B.

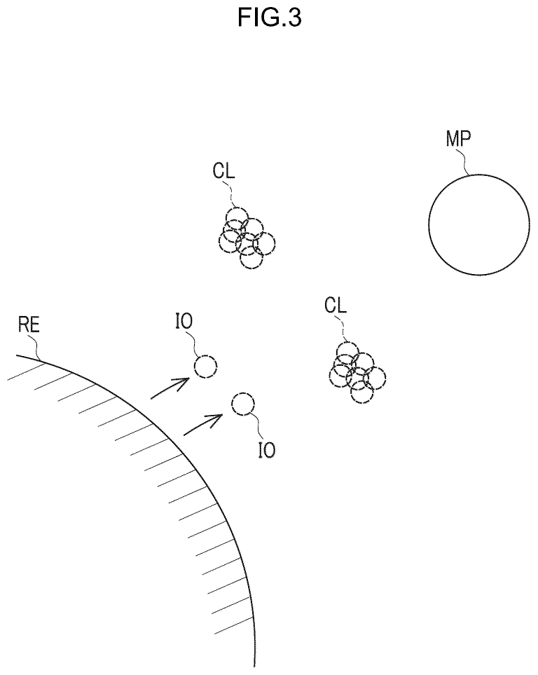

FIG. 3 is a schematic view conceptually illustrating the colloidal particles CL. An ion exchange resin RE schematically represents a cation exchange resin and an anion exchange resin provided to the non-regenerative mixed bed ion exchange resin device 24. The ions IO of an organic substance derived from a functional group of the ion exchange resin may be peeled off from the surface of the ion exchange resin RE, and when the ions IO are aggregated in a liquid, the colloidal particles CL may be formed. The colloidal particles CL may be formed in water after the ions IO are desorbed from the ion exchange resin RE, but may be formed on the ion exchange resin RE or at a contact portion with water in the secondary pure water device 13, for example, on the filtration membrane of the ultrafiltration membrane device 25 or the surface of a pipe. Here, such a colloidal particle CL as described above is a concept including a monomer, a dimer, a trimer, and a polymer. The particle size of the colloidal particle CL is considered to be in a range from a size of several nanometers to several tens of nanometers or less. The particle size of the colloidal particle CL includes a size similar to that of such a particle MP as described above. As described above, unlike the ions IO, the colloidal particles CL do not completely dissolve into a liquid, and have both properties of non-particulate properties such as the ions IO and particulate properties such as the particles MP.

In view of such properties of the colloidal particles CL, the inventors have found that the colloidal particles CL can be measured based on the respective measurement values of the optical particle counter 31B and the acoustic particle counter 31A. That is, the inventors have found, through the experiments based on a hypothesis, that the acoustic particle counter 31A can detect the colloidal particles CL in addition to the particles MP, and that the colloidal particles CL can be quantitatively grasped by the combination of the optical particle counter 31B and the acoustic particle counter 31A.

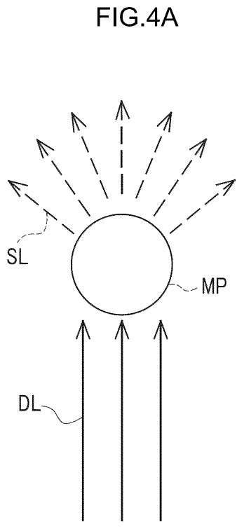

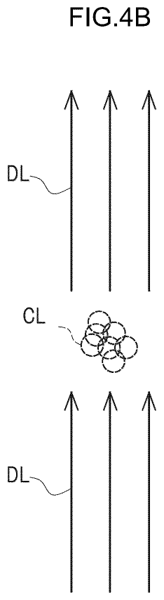

FIGS. 4A and 4B are schematic view conceptually illustrating the reason for the difficulty in detection of the colloidal particles CL by the optical particle counter 31B. As illustrated in FIG. 4A, the optical particle counter 31B emits laser light DL to such a particle MP as described above and detects scattered light SL scattered from the particle MP, thereby detecting the particle MP. However, the colloidal particles CL strongly exhibit non-particulate properties optically, and have a very small refractive index as compared with the particles MP. Therefore, as illustrated in FIG. 4B, even when such a colloidal particle CL as described above is irradiated with the laser light DL, scattering hardly occurs. It is considered that the optical particle counter 31B can detect the particles MP for such a reason, but cannot detect the colloidal particles CL.

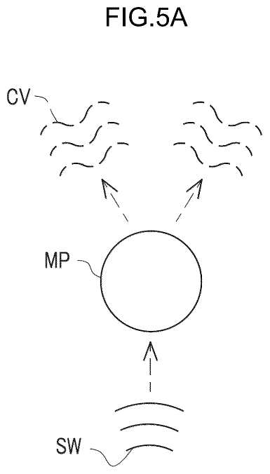

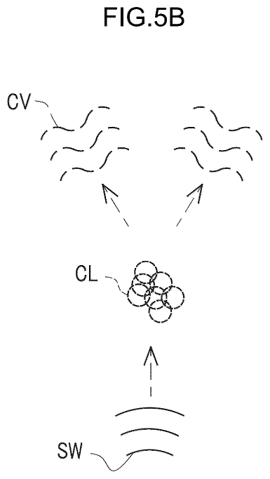

FIGS. 5A and 5B are schematic view illustrating the reason that the colloidal particles CL can be detected by the acoustic particle counter 31A. As illustrated in FIG. 5A, the acoustic particle counter 31A emits a sound wave SW to such a particle MP as described above, and detects the pressure based on the cavitation CV of the liquid generated by the vibration of the particle MP having received the sound wave SW. Such a colloidal particle CL as described above does not dissolve in the liquid and is present as an entity having an outer edge similarly to the particle MP. Therefore, as illustrated in FIG. 5B, when a sound wave SW is emitted to the colloidal particle CL, the colloidal particle CL vibrates similarly to the particle MP, whereby the cavitation CV of the liquid is generated. It is considered that the acoustic particle counter 31A can also detect the colloidal particle CL by detecting the pressure based on the cavitation CV.

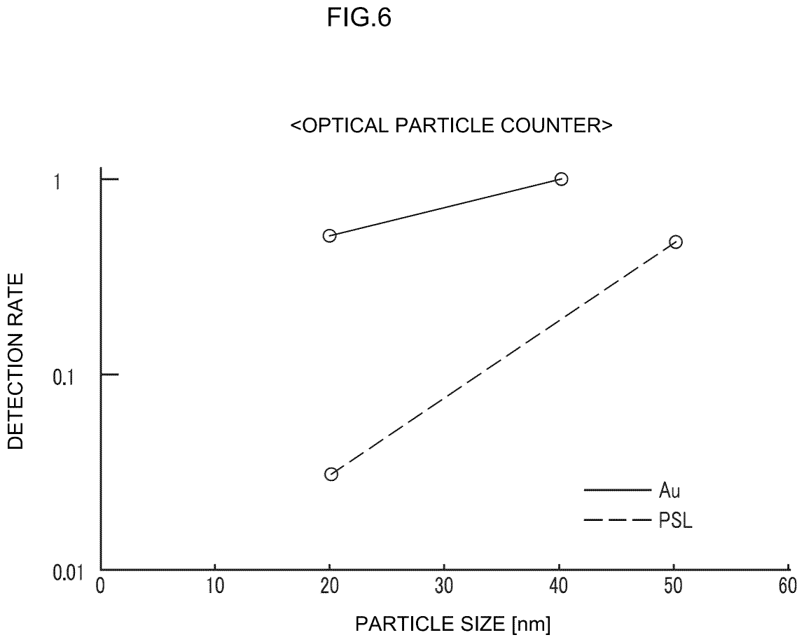

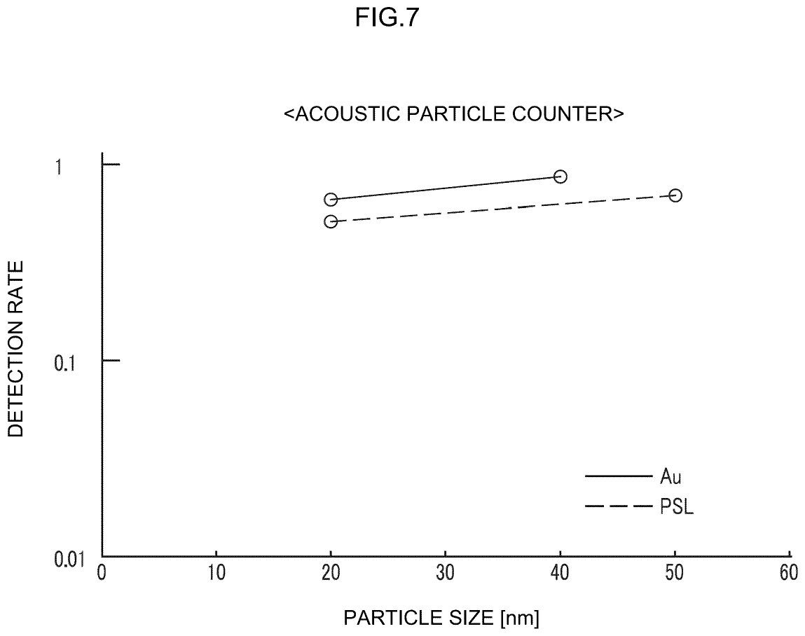

FIGS. 6 and 7 illustrate the experimental results for verifying the above hypothesis. The experiments were performed by adding colloidal gold standard particles and polystyrene latex (PSL) standard particles online to ultrapure water, and installing a particle counter at the following stage of the ultrafiltration membrane device 25 to measure the amount of particles. As the colloidal gold standard particles, a standard colloidal gold manufactured by BBI Solutions was used, and as the PSL standard particles, standard latex particles manufactured by Thermo Fisher Scientific Inc. were used. The colloidal gold standard particles are the models of particles MP, and the PSL standard particles are the models of colloidal particles. Hereinafter, the colloidal gold standard particles are referred to as colloidal gold particles, and are denoted by AU in the graphs. The PSL standard particles are referred to as latex spheres and are denoted by PSL in the graphs. As the acoustic particle counter 31A and the optical particle counter 31B, the above-described products were used.

FIG. 6 is a graph illustrating the experimental result obtained from the experiment in which the colloidal gold particles (Au) as the models of particles MP, and the latex spheres (PSL) as the models of colloidal particles CL were detected by the optical particle counter 31B. FIG. 7 is a graph illustrating the experimental result obtained from the experiment in which the colloidal gold particles (Au) and the latex spheres (PSL) were detected by the acoustic particle counter 31A. In each of FIGS. 6 and 7, the horizontal axis represents the particle size [nm], and the vertical axis represents the detection rate. In FIGS. 6 and 7, there is no data indicating that the particle size is less than 20 nm, because the detection limit of each of the acoustic particle counter 31A and the optical particle counter 31B is 20 nm. As the acoustic particle counter 31A and the optical particle counter 31B, the above-described products were used.

The latex spheres (PSL) are organic substances, and it is presumed that as the particle size is finer, the properties of the colloid are more enhanced. On the other hand, it is presumed that this tendency is less likely to occur in the colloidal gold particles (Au) due to their properties as metals. As illustrated in FIG. 6, it is found that the optical particle counter 31B has a deviation in the detection rate between the colloidal gold particles (Au) and the latex spheres (PSL) and thus the detection rate of the latex spheres (PSL) is lower than that of the colloidal gold particles (Au). The difference increases as the particle size is smaller. Thus, it can be found that it is more difficult to perform detection by the optical particle counter 31B as a non-particulate property is more enhanced. The experimental result is consistent with the hypothesis as illustrated in FIGS. 4A and 4B that it is difficult to detect the colloidal particle CL having a refractive index lower than that of the particle MP because the scattered light SL is hardly generated.

On the other hand, as illustrated in FIG. 7, it is found that the acoustic particle counter 31A has no deviation in the detection rate between the colloidal gold particles (Au) and the latex spheres (PSL) and thus the latex spheres (PSL) are detected as much as the colloidal gold particles (Au). The experimental result is consistent with the hypothesis as illustrated in FIGS. 5A and 5B that the colloidal particle CL exhibits an acoustically particulate property and generates the cavitation CV due to receiving of the sound wave SW.

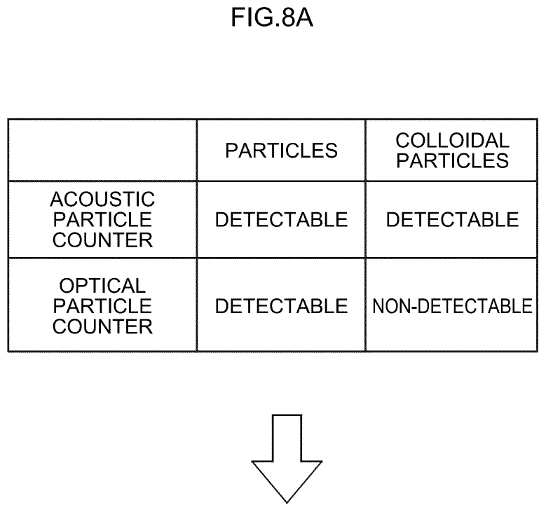

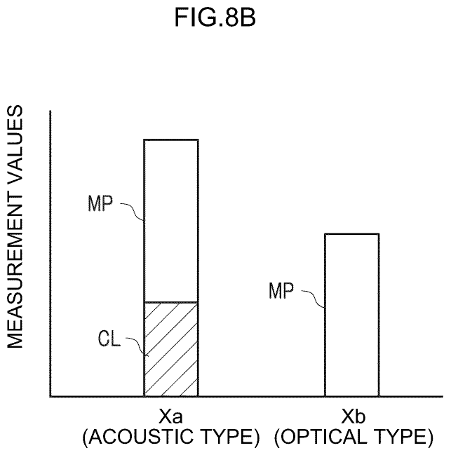

These results are organized as illustrated in FIGS. 8A and 8B. That is, as indicated in FIG. 8A, the acoustic particle counter 31A can detect both the particles MP and the colloidal particles CL, whereas the optical particle counter 31B can detect the particles MP but cannot detect the colloidal particles CL. Therefore, as illustrated in FIG. 8B, the measurement value Xa of the acoustic particle counter 31A includes both the measurement value of the particles MP and the measurement value of the colloidal particles CL. On the other hand, the measurement value Xb of the optical particle counter 31B does not include the measurement value of the colloidal particles CL but includes the measurement value of the particles MP.

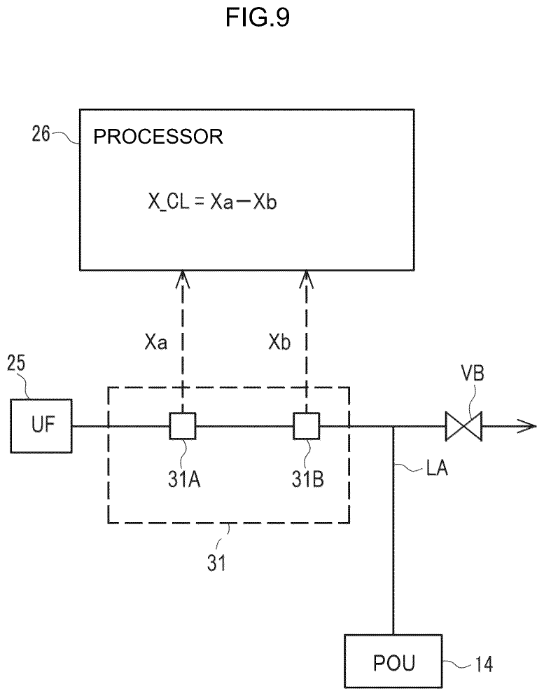

Therefore, as illustrated in FIG. 9, in the processor 26, the measurement value X_CL of the colloidal particles CL can be obtained by subtracting the measurement value Xb of the optical particle counter 31B from the measurement value Xa of the acoustic particle counter 31A. Similarly to the measurement values Xa and Xb, the measurement value X_CL is the number of colloidal particles CL per unit volume. In such a manner, the processor 26 can quantitatively grasp the colloidal particles CL.

Next, a method of removing the colloidal particles CL thus measured will be described. Because many of the colloidal particles CL have a particle size smaller than that of the particles MP, it has been considered that the removal of the colloidal particles CL is difficult even with the ultrafiltration membrane device 25. Further, focusing on the fact that the colloidal particles CL are aggregates of the ions IO, the removal of the colloidal particles CL by capturing by an electrical method was attempted. However, because the concentration of the colloidal particles CL was low, the contact probability of the ions IO with a substance that captures the ions IO was low, and thus it was difficult to capture the colloidal particles CL even by the electrical method.

Therefore, the inventors have found a method of removing the colloidal particles CL from treatment-target water by controlling the operating conditions of the ultrafiltration membrane device 25. Specifically, the method includes operating the ultrafiltration membrane device 25 with the water recovery rate set at less than 80%, as a method of managing operation of the ultrapure water production apparatus. This method enabled prevention of the colloidal particles CL smaller than the pore size of the ultrafiltration membrane from passing through the ultrafiltration membrane as described below. The control of the water recovery rate is performed by the processor 26 controlling the respective opening degrees of the valves VA and VB and the respective driving conditions of the water supply pump P1 and the booster pump P2. “The water recovery rate” is defined by a ratio of a flow rate of treated water recovered from the ultrafiltration membrane device to which the treatment-target water has been supplied to a flow rate of treatment-target water supplied to the ultrafiltration membrane device.

First, it has been conventionally considered that in an ultrafiltration membrane device, particles MP larger than the pore size of the ultrafiltration membrane are captured water and removed from water, namely, filtration mainly is advanced by the mechanism of surface filtration. However, the colloidal particles CL observed in terminal ultrapure water are often smaller than the pore size of the ultrafiltration membrane, and for this reason, it is considered that the colloidal particles CL cannot be removed by such a surface filtration as described above. As the result of the study, the present inventors conducted an experiment assuming that filtration of the colloidal particles CL is advanced not by the mechanism of surface filtration but by the mechanism of depth filtration. As a result, the present inventors have found that the colloidal particles CL are removed by the ultrafiltration membrane device 25 by filtering in a cross-flow manner under the condition of the above-described water recovery rate. The reason is not necessarily clear, but is considered as follows.

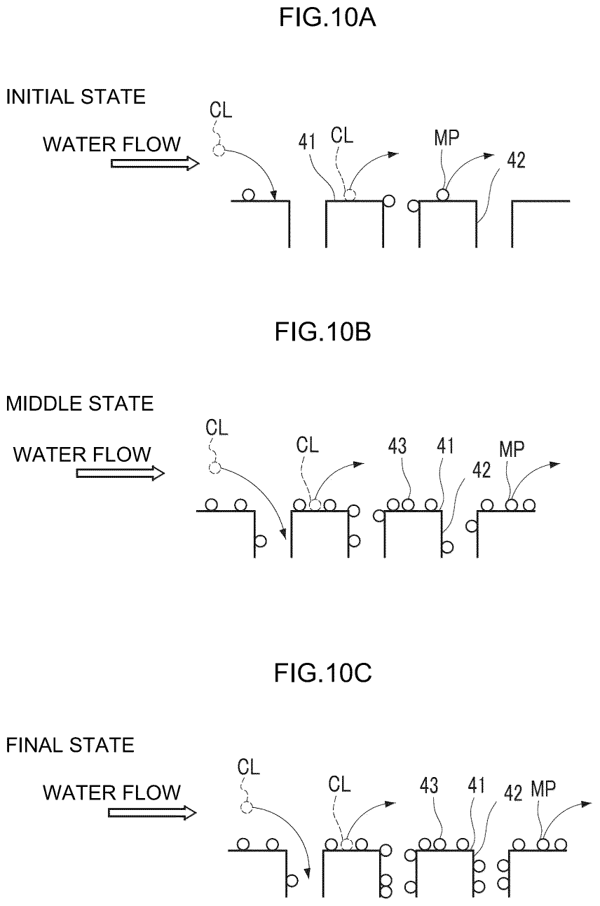

FIGS. 10A to 10C schematically illustrates the states in which the particles MP and the colloidal particles CL are captured on the filtration membrane in membrane filtration in the present embodiment. From above, FIGS. 10A, 10B, and 10C are schematic views illustrating, respectively, the initial state of filtration, the advanced state (in other words, middle state) of filtration, and the further advanced state (in other words, final state) of filtration.

In FIGS. 10A to 10C, the rectangles represent the membrane surface 41 and the pore inner wall 42 of the filtration membrane, and the small circles with solid lines represent the particles MP. The small circles with broken lines represent the colloidal particles CL. The thick arrows each indicate a water flow, and the thin arrows each indicate the movement of the particles MP.

First, the mechanism by which the particles MP are captured on the filtration membrane will be described. Two locations where the particles MP are captured and adsorbed are considered: the membrane surface 41 and the pore inner wall 42. When the filtration membrane is new or nearly new, the number of the particles MP already adsorbed on the membrane surface 41 or the pore inner wall 42 of the filtration membrane is smaller. Therefore, both the membrane surface 41 and the pore inner wall 42 can adsorb the particles MP. Because the treatment-target water first comes into contact with the membrane surface 41, as illustrated in FIG. 10A, the membrane surface 41 mainly adsorbs the particles MP. Thereafter, when the adsorption of the particles MP on the membrane surface 41 is advanced, the adsorption of the particles MP on the pore inner wall 42 is gradually advanced as illustrated in FIG. 10B.

As the membrane filtration is further advanced and the adsorption of the particles MP on the ultrafiltration membrane is advanced, the amount of the particles MP adsorbed on the membrane surface 41 or the pore inner wall 42 increases. This increase makes it difficult for the particles MP to be newly adsorbed on the membrane, thereby the particles MP permeate the membrane as illustrated in FIG. 10C. In this case, the removal rate of the particles MP decreases.

Here, the particles MP are repeatedly adsorbed on the membrane surface 41 and desorbed from the membrane surface 41 of the ultrafiltration membrane in the process of membrane filtration. Therefore, if the membrane filtration is performed in a cross-flow manner to increase the flow rate along the membrane surface 41 and prevent an increase in the concentration of the particles MP on the membrane surface 41, the initial state as illustrated in FIG. 10A can be maintained. As a result, it is considered that the particles MP smaller than the pore size of the ultrafiltration membrane can be prevented from passing through the pores of the ultrafiltration membrane and thus a high removal rate can be maintained.

The behavior of the colloidal particles CL is similar to that of the particles MP smaller than the pore size of the ultrafiltration membrane, and it is considered that the adsorption on the membrane surface 41 and the desorption from the membrane surface 41 of the ultrafiltration membrane are repeated in the process of membrane filtration. Therefore, it is considered that prevention of an increase in the concentration of the colloidal particles CL adhering to the membrane surface 41 enables prevention of the colloidal particles CL smaller than the pore size of the ultrafiltration membrane from passing through the pores of the ultrafiltration membrane and as a result, the colloidal particles CL can be removed.

As an example, the method of maintaining the initial state illustrated in FIG. 10A is a method of operation with the water recovery rate set at less than 80%. Usually, the water recovery rate is often set at 80% or more, but the flow rate along the membrane surface 41 can be increased by intentionally lowering the water recovery rate to less than 80%, that is, increasing the flow rate on the concentration side. As a result, the amount of the particles MP and the colloidal particles CL deposited on the membrane surface 41 is reduced, so that it is considered that the initial state illustrated in FIG. 10A can be maintained.

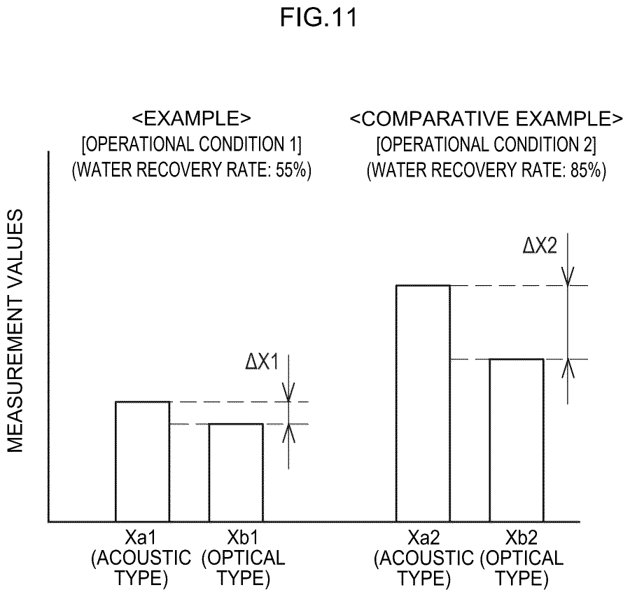

FIG. 11 is a graph indicating the experimental result of an attempt to remove the colloidal particles CL by controlling the water recovery rate, as an exemplary method of using the ultrapure water production apparatus 10 including the measurement device 31 of the embodiment. In the operating condition 1 of the ultrafiltration membrane device 25 in Example 1, the water recovery rate is 55%, and in the operating condition 2 in Example 2, the water recovery rate is 85%. The differential pressure between the permeation side and the concentration side of the filtration membrane is 1 kgf/cm². As a filtration membrane, Microsa (registered trademark) OAT-6036 (molecular weight cutoff: 6000) manufactured by Asahi Kasei Corporation was used. As the mixed bed ion exchange resin of the non-regenerative mixed bed ion exchange resin device 24, “N-LITE (registered trademark) MBSP” manufactured by Nomura Micro Science Co., Ltd. was used, and the space velocity (SV) was 40 (1/h). In the experiment, first, the water recovery rate of the ultrafiltration membrane device 25 was set at 85%, the secondary pure water device 13 was operated with this setting, and data is measured. Thereafter, the water recovery rate was set at 55%, and the secondary pure water device 13 was operated for 30 days. After the data was stabilized, the measurement was performed, and the measurement result was obtained. As the secondary pure water device 13 used in the experiment, a secondary pure water device 13 having the same configuration as in FIG. 2 was used except that the ultraviolet irradiator 22 and the membrane degassing device 23 were not provided.

In Example 1, the measurement value of the acoustic particle counter 31A is Xa1, the measurement value of the optical particle counter 31B is Xb1, and the difference is ΔX1 that is the number of colloidal particles CL per unit volume in Example 1. In Example 2, the measurement value of the acoustic particle counter 31A is Xa2, the measurement value of the optical particle counter 31B is Xb2, and the difference is ΔX2 that is the number of colloidal particles CL per unit volume in Comparative Example. More specific numerical values are indicated in Table 1 below.

TABLE 1

| | | | | | Colloidal | |
|---|---|---|---|---|---|---|
| | Acoustic type | | Optical type | | particles | |
| | (Number/L) | | (Number/L) | | (Xa − Xb) | |
| Example 1 | Xa1 | 256 | Xb1 | 240 | ΔX1 | 16 |
| Example 2 | Xa2 | 650 | Xb2 | 432 | ΔX2 | 218 |

As indicated in Table 1, the number ΔX1 of the colloidal particles CL in Example 1 was decreased to 1/10 or less as compared with ΔX2 in Example 2.

It was verified that the colloidal particles CL are removed by setting the water recovery rate at less than 80% as in Example 1.

The water recovery rate is at less than 80%, but when the water recovery rate is less than 50%, the water recovery rate is too small, and thus the practicability is deteriorated. From the viewpoint of improving the removal rate of the colloidal particles CL to efficiently obtain ultrapure water, the water recovery rate of the ultrafiltration membrane device 25 is preferably 50% or more and less than 80%.

As described above, the ultrapure water production apparatus 10 according to the technology of the present disclosure is an ultrapure water production apparatus 10 including the secondary pure water device 13 including the water supply pump P1 (that is to say, an exemplary water supply pump), the non-regenerative mixed bed ion exchange resin device 24 (that is to say, an exemplary ion exchanger), and the ultrafiltration membrane device 25 provided in this order, the secondary pure water device 13 being configured to remove impurities from primary pure water, in which the combination of the acoustic particle counter 31A and the optical particle counter 31B, which corresponds to a first combination, is disposed at the downstream side of the ultrafiltration membrane device 25. Such an ultrapure water production apparatus 10 can be used for a method of measuring colloidal particles CL. The ultrapure water production apparatus 10 is used in an ultrapure water production method according to the technology of the present disclosure.

As the water pump, the water supply pump P1 may be used, or the booster pump P2 may be further provided as in the above embodiment.

Because the ultrapure water production apparatus 10 according to the technology of the present disclosure includes the combination of the acoustic particle counter 31A and the optical particle counter 31B, the colloidal particles CL in the treatment-target water can be quantitatively grasped as described with reference to FIGS. 8A, 8B and 9.

The ultrapure water production apparatus 10 according to the technology of the present disclosure further includes the processor 26 configured to control the water recovery rate of the ultrafiltration membrane device 25. Therefore, as illustrated in FIGS. 10A to 10C and indicated in FIG. 11, the colloidal particles CL contained in the ultrapure water can be removed by controlling the water recovery rate.

In the present embodiment, the comparison at the same detection limit was performed between the acoustic particle counter 31A and the optical particle counter 31B, but the detection limits are not necessarily the same. In the case of different detection limits, a difference between the measurement value of the acoustic particle counter 31A and the measurement value of the optical particle counter 31B cannot be obtained. However, the tendency of the increase or decrease of the colloidal particles CL can be grasped from the tendency of both the measurement values. Further, use of a conversion coefficient between the measurement value of the acoustic particle counter 31A and the measurement value of the optical particle counter 31B enables estimation of the amount of the colloidal particles CL in advance. For example, when the same tendency as in FIG. 6 is obtained for each particle size in advance using latex spheres (PSL) various in particle size, the amount of the colloidal particles CL can be obtained by a conversion method using a conversion coefficient with the tendency even when the detection limits are different.

### Modification

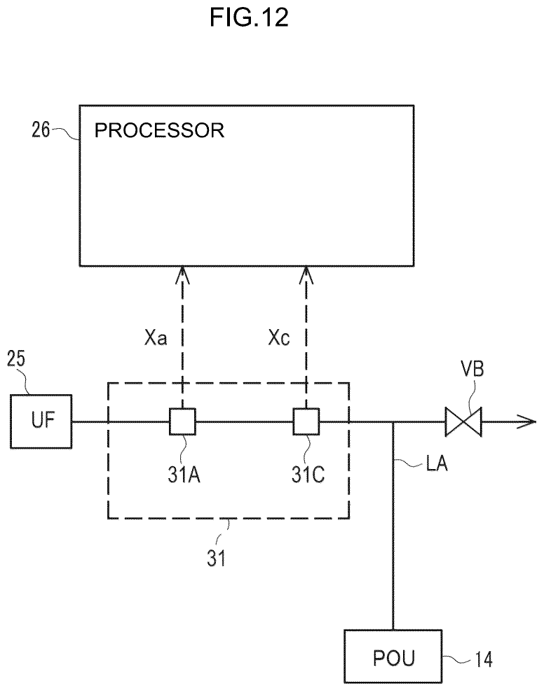

In the modification illustrated in FIG. 12, the combination of measuring instruments used for measuring colloidal particles CL is different from the combination of the embodiment illustrated in FIG. 9. The other configurations are similar to those of the embodiment, and thus only the differences will be described below.

The present modification is based on the result of the study under the hypothesis that a TOC meter 31C can be used for measuring the amount of colloidal particles CL. That is, the TOC meter 31C is a measuring instrument that measures the total carbon amount of organic substances in water, and because the colloidal particles CL contain organic substances, at least part of the measurement value of the TOC meter 31C should correspond to the amount of the colloidal particles CL. Further, a hypothesis can be assumed that in ultrapure water, for example, the colloidal particles of a single element such as a silica colloid, an iron colloid, and an organic colloid are not present in a mixed state, but colloidal particles CL composed of a plurality of elements are present, and furthermore, the component ratio in the colloidal particles CL does not change depending on the individual of the colloidal particles CL and is substantially constant.

Then, a hypothesis is established that there should be some relationship between the value of the TOC meter 31C and the amount of the colloidal particles CL obtained by the method of the embodiment (i.e., ΔX=Xa−Xb). The present modification is a finding found based on this hypothesis.

In the above embodiment, the colloidal particles CL are measured based on the measurement value of the acoustic particle counter 31A and the measurement value of the optical particle counter 31B. However, in the modification illustrated in FIG. 12, the combination of the measuring instruments of the acoustic particle counter 31A and the TOC meter 31C, which corresponds to a second combination, is used to measure the colloidal particles CL, based on the measurement value of the acoustic particle counter 31A and the measurement value of the TOC meter 31C. However, in the case of the modification, it is difficult to quantify the colloidal particles CL, but the colloidal particles CL can be measured to such an extent that the tendency of increase or decrease of the colloidal particles CL is grasped.

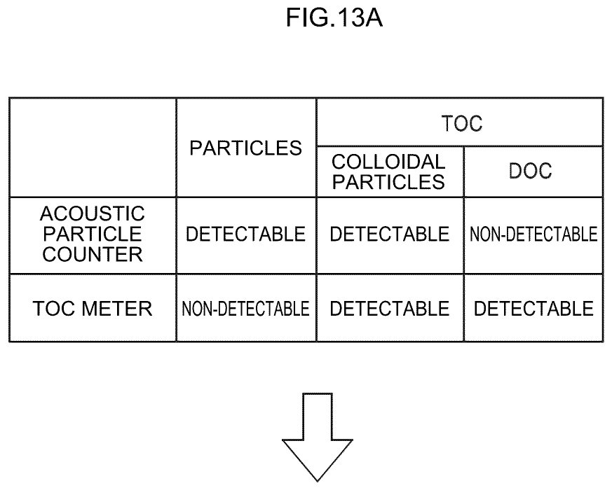

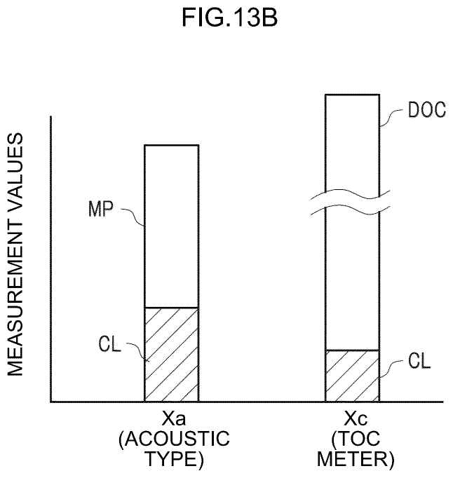

In FIGS. 13A and 13B, as indicated in FIG. 13A, the acoustic particle counter 31A can detect both the particles MP and the colloidal particles CL, but cannot detect the dissolved organic carbon (DOC) dissolved in water. On the other hand, the TOC meter 31C can detect the DOC. Furthermore, as described above, the TOC meter 31C can detect the amount of particulate organic carbon (that is to say, the amount of particulate organic carbon that is not dissolved in the treatment-target water) in addition to the DOC. The proportion of the amount of particulate organic carbon contained in the total carbon amount (TOC) is also very small, but most of the amount of particulate organic carbon is considered to include colloidal particles CL. As described above, the colloidal particles CL can be detected also by the TOC meter 31C.

Then, as indicated in FIG. 13B, the measurement value Xa of the acoustic particle counter 31A includes the particles MP and the colloidal particles CL, and the measurement value Xc of the TOC meter 31C includes the DOC and the colloidal particles CL. As a result of examining the relationship between the amount (that is to say, Xa-Xb) of the colloidal particles CL obtained from the difference between the measurement value Xa of the acoustic particle counter 31A and the measurement value Xb of the optical particle counter 31B and Xc, it has been found that there is generally the following relationship. That is, when there is no change in the measurement value Xc and the measurement value Xa increases or decreases, it is considered that the amount of the particles MP increases or decreases. On the other hand, there is no change in the measurement value Xa and the measurement value Xc increases or decreases, it is considered that the amount of the DOC increases or decreases. Furthermore, when both the measurement values Xa and Xc increase or decrease, it is considered that there is a high possibility that the amount of the colloidal particles CL increases or decreases. The processor 26 can grasp the tendency of the increase or decrease of the amount of the colloidal particles CL, based on the fluctuations of the measurement values Xa and Xc. As described above, in the technology of the present disclosure, the concept of “measuring colloidal particles” includes grasping the tendency of increase or decrease of the colloidal particles CL in addition to quantitatively grasping the colloidal particles CL.

So far, it has been difficult to quantitatively grasp the colloidal particles CL and to grasp the tendency of increase and decrease of the colloidal particles CL. Therefore, even if it is not possible to quantitatively grasp the colloidal particles CL, there is a sufficient merit in practical use only by being able to grasp the tendency of increase and decrease of the colloidal particles CL.

For example, in the case of operation of the ultrafiltration membrane device 25 with the water recovery rate set at 55% as indicated in FIG. 11 after the decrease of the amount of DOC to a substantially constant amount, if it can be confirmed that both the measurement values Xa and Xc have decreased, it is considered that the probability that the amount of colloidal particles CL has decreased is high. In this case, the processor 26 may be able to quantitatively grasp the amount of the colloidal particles CL by executing a predetermined calculation based on the two measurement values Xa and Xc.

Here, the measurement value of the TOC meter 31C indicates concentration, and the unit is [ppb]. On the other hand, the measurement value of the acoustic particle counter 31A indicates the number per unit volume, and the unit is [number/L]. Therefore, the colloidal particles CL cannot be quantified simply by obtaining a difference between the measurement value Xa of the acoustic particle counter 31A and the measurement value Xb of the optical particle counter 31B, by using the combination of the acoustic particle counter 31A and the optical particle counter 31B. However, use of an arithmetic equation including the measurement value Xc of the TOC meter 31C and a coefficient for converting the concentration into the number enables estimation of the approximate amount of the colloidal particles CL.

Even in the case of the modification, a method of removing the colloidal particles CL by setting the water recovery rate of the ultrafiltration membrane device 25 at less than 80% can be applied.

Table 2 below indicates the experimental results of the modification. In Example 1 of Table 2, the TOC was measured with the TOC meter 31C at the same timing of the measurement in Example 1 of Table 1. Similarly to Example 1 of Table 2, in Example 2 of Table 2, the TOC was measured with the TOC meter 31C at the same timing of the measurement in Example 2 of Table 1. As the TOC meter 31C, Sievers M500e manufactured by SUEZ was used.

TABLE 2

| | | | | | Colloidal | |
|---|---|---|---|---|---|---|
| | Acoustic type | | TOC | | particles | |
| | (Number/L) | | (ppb) | | (Xa − Xb) | |
| Example 1 | Xa1 | 256 | Xc1 | 0.14 | ΔX1 | 16 |
| Example 2 | Xa2 | 650 | Xc2 | 0.4 | ΔX2 | 218 |

| | | |
|---|---|---|
| Rate of increase | −61 | −65 |
| or decrease (%) | | |

In Table 2, when Example 1 and Example 2 are compared, from the difference (that is to say, ΔX1 and ΔX2) between the measurement value Xa of the acoustic particle counter 31A and the measurement value Xb of the optical particle counter 31B, it can be found that the colloidal particles CL decrease in Example 1. However, the measurement value Xa1 of Example 1 also decreases in comparison with the measurement value Xa2 of Example 2, and the measurement value Xc1 of Example 1 also decreases in comparison with the measurement value Xc2 of Example 2. Because the both measurement values Xa and Xc decrease in this manner, it can be determined from the tendency that the colloidal particles CL decrease.

Table 3 below indicates the comparison between Example 2 and Example 3. In Table 3, Example 2 is similar to Example 2 of Table 2. Example 3 is the same as Example 2 except that the space velocity (SV) of the non-regenerative mixed bed ion exchange resin device 24 was changed from 24 (1/h) to 200 (1/h) among the conditions of Example 2. Example 3 is the case of attempting a decrease of the TOC by increasing the SV.

TABLE 3

| | | | | | COLLOIDAL | |
|---|---|---|---|---|---|---|
| | Acoustic type | | TOC | | PARTICLES | |
| | (Number/L) | | (ppb) | | (Xa − Xb) | |
| Example 3 | Xa3 | 750 | Xc3 | 0.22 | ΔX3 | 220 |
| Example 2 | Xa2 | 650 | Xc2 | 0.4 | ΔX2 | 218 |

| | | |
|---|---|---|
| Rate of increase | 15 | −45 |
| or decrease (%) | | |

In the comparison between Example 2 and Example 3, there is no significant increase or decrease in the amount of the colloidal particles CL and the amount of colloidal particles CL are also equal to each other. The measurement values Xa2 and Xa3 of the acoustic particle counter 31A are also substantially equal to each other. On the other hand, in comparison between the measurement values Xc2 and Xc3 of the TOC meter 31C, the measurement value Xc3 decreases in comparison with the measurement value Xc2. Therefore, Table 3 corresponds to a case where there is no change in the measurement value Xa and the measurement value Xc decreases. In this case, it can be determined that the decrease in TOC is due to the decrease in DOC from the relationship between the increase or decrease in the measurement values Xa and Xc.

As described above, as indicated in Table 2 and Table 3, it is found that there is a correlation between the amount (that is to say, Xa-Xb) of the colloidal particles CL obtained from the difference between the measurement value Xa of the acoustic particle counter 31A and the measurement value Xb of the optical particle counter 31B and the combination of the measurement value Xa of the acoustic particle counter 31A and the measurement value Xc of the TOC meter 31C. Therefore, the use of the combination of the acoustic particle counter 31A and the TOC meter 31C enables a grasp of at least the tendency of the increase or decrease of the colloidal particles CL.

In the case of using the optical particle counter 31B instead of the acoustic particle counter 31A in Modification 1, the same result as in Modification 1 was not obtained, and thus no clear relationship was found between the amount (that is to say, Xa-Xb) of the colloidal particles CL and the measurement value Xc of the TOC meter 31C. Therefore, in order to measure the colloidal particles CL using the present disclosure, the acoustic particle counter 31A is essential, and it has been confirmed that it is effective to use the optical particle counter 31B or the TOC meter 31C in a form of supplementing the acoustic particle counter 31A.

As described above, the ultrapure water production apparatus 10 according to the modification of the technology of the present disclosure has the combination of the acoustic particle counter 31A and the TOC meter 31C, which corresponds to the second combination. The TOC meter 31C is a measuring instrument that can be said to be essential in the field of ultrapure water production and has been widely used so far. Therefore, according to the modification, the TOC meter 31C used so far can be effectively used for measuring the colloidal particles CL. As a matter of course, in the case of the modification, the optical particle counter 31B may be omitted.

In addition, the measurement device 31 of the first combination or the second combination included in the ultrapure water production apparatus 10 according to the technology of the present disclosure can be used for managing operation of the ultrapure water production apparatus 10 in addition to the measurement of the colloidal particles CL. That is, in a case where the measurement device 31 determines that the amount of the colloidal particles CL has changed or the amount of the colloidal particles CL is highly likely to have changed, the ultrapure water production apparatus 10 may have some cause. For example, in the case of an increase in the amount of the colloidal particles CL, if the increase is caused by a change in the operating condition, the deterioration in water quality can be improved by returning the operating condition. If it can be determined that the defect is caused by the failure of some kind of device, the problem can be solved by elimination of the failure of the device. If the operating conditions of the ultrapure water production apparatus 10 are changed and the amount of the colloidal particles CL is changed, it is also possible to find the optimum operating conditions. This tendency can be grasped by monitoring the change in the measured values over time using the first combination of the acoustic particle counter 31A and the optical particle counter 31B or the second combination of the acoustic particle counter 31A and the TOC meter 31C, so that an excellent effect that such operation management of the ultrapure water production apparatus 10 described above can be easily performed can be obtained.

In the case where the first combination of the two measuring instruments of the acoustic particle counter 31A and the optical particle counter 31B in the embodiment or the second combination of the two measuring instruments of the acoustic particle counter 31A and the TOC meter 31C in the modification is provided, the disposition of the two measuring instruments can be variously changed. For example, the two measuring instruments may be disposed in series, or may be disposed in parallel in a branch path and another branch path branching from a single pipeline.

The installation positions of the two measuring instruments may be on the downstream side or may be on the upstream side of the ultrafiltration membrane device 25. On the downstream side of the ultrafiltration membrane device 25, the amount of the colloidal particles CL of treated water of the ultrafiltration membrane device 25, namely, the amount of the colloidal particles CL of the ultrapure water to be produced, can be directly measured. On the other hand, on the upstream side of the ultrafiltration membrane device 25, the amount of the colloidal particles CL of the water supplied to the ultrafiltration membrane device 25 can be also measured. In the case of installation on the upstream side and the downstream side of the ultrafiltration membrane device 25, the amount of the colloidal particles CL removed by the ultrafiltration membrane device 25 can be directly measured.

In the embodiment, the processor 26 may include a single piece of hardware or a plurality of pieces of hardware, and the type of hardware is not limited. For example, the processor 26 may include a central processing unit (CPU), a programmable logic device such as a field programmable gate array (FPGA), or a specific circuit such as an application specific integrated circuit (ASIC).

The technology of the present disclosure may appropriately combine the various embodiments and/or various modifications. Further, it is a matter of course that various configurations can be adopted without departing from the gist without being limited to the embodiments.

The contents described and illustrated above are detailed descriptions of parts according to the technology of the present disclosure, and thus are merely examples of the technology of the present disclosure. For example, the above description regarding the configurations, functions, operations, and effects is a description regarding an example of the configurations, functions, operations, and effects of the parts according to the technology of the present disclosure. Therefore, it is needless to say that unnecessary portions may be removed, new elements may be added, or replacement may be made to the above described and illustrated contents without departing from the gist of the technology of the present disclosure. Furthermore, in order to avoid complication and to facilitate understanding of the parts according to the technology of the present disclosure, in the contents described and illustrated above, description regarding technical common sense and others that do not require any particular description in enabling implementation of the technology of the present disclosure is not given.

The disclosure of Japanese Patent Application No. 2025-020996, filed on Feb. 12, 2025, is incorporated herein by reference in its entirety. All documents, patent applications, and technical standards that are described in the present specification are incorporated herein by reference to the same extent as if each individual document, patent application, and technical standard was specifically and individually indicated to be incorporated by reference.

1. An ultrapure water production apparatus comprising a secondary pure water device including: a water supply pump, an ion exchanger, and an ultrafiltration membrane device in this order, the secondary pure water device being configured to remove an impurity from primary pure water, wherein:
at least one of a first combination of an acoustic particle counter and an optical particle counter or a second combination of the acoustic particle counter and a total organic carbon (TOC) meter is disposed at at least one of an upstream side or a downstream side of the ultrafiltration membrane device.

2. The ultrapure water production apparatus according to claim 1, further comprising a processor configured to control a water recovery rate of the ultrafiltration membrane device.

3. A method of producing ultrapure water using the ultrapure water production apparatus according to claim 1.

4. A method of managing operation of an ultrapure water production apparatus comprising a secondary pure water device including: a water supply pump, an ion exchanger, and an ultrafiltration membrane device in this order, the secondary pure water device being configured to remove an impurity from primary pure water, the method comprising:
using at least one of a first combination of an acoustic particle counter and an optical particle counter or a second combination of the acoustic particle counter and a total organic carbon (TOC) meter, the at least one of the first or second combination being disposed at a downstream side of the ultrafiltration membrane device; and
managing operation of the ultrapure water production apparatus, based on measurement values acquired from the at least one of the first or second combination.

5. A method of measuring colloidal particles in a secondary pure water device including: a water supply pump, an ion exchanger, and an ultrafiltration membrane device in this order, the secondary pure water device being configured to remove an impurity from primary pure water, the method comprising:
using at least one of a first combination of an acoustic particle counter and an optical particle counter or a second combination of the acoustic particle counter and a total organic carbon (TOC) meter, the at least one of the first or second combination being disposed at a downstream side of the ultrafiltration membrane device; and
measuring colloidal particles, based on measurement values acquired from the at least one of the first or second combination.
